(19) United States
(12) Patent Application Publication
(10) Pub. No.: US 2013/0150591 A1
(43) Pub. Date: Jun. 13, 2013

(54) METHOD FOR PURIFYING N-ALKYL-SUBSTITUTED PYRROLIDONES BY HYDROGENATION

(71) Applicants: Rolf PINKOS, Bad Duerkheim (DE); Thomas Vogler, Ludwigshafen (DE); Karl Ott, Plankstadt (DE)

(72) Inventors: Rolf PINKOS, Bad Duerkheim (DE); Thomas Vogler, Ludwigshafen (DE); Karl Ott, Plankstadt (DE)

(21) Appl. No.: 13/705,654

(22) Filed: Dec. 5, 2012

Related U.S. Application Data
(60) Provisional application No. 61/567,659, filed on Dec. 7, 2011.

(51) Int. Cl. C07D 207/27

(52) U.S. Cl. CPC C07D 207/27
USPC 548/555

(57) ABSTRACT

The invention relates to a method for purifying N-alkyl-substituted pyrrolidones which have contaminants with a higher degree of oxidation, by hydrogenation of the same and distillation.

The invention relates to a method for purifying N-alkyl-substituted pyrrolidones which have contaminants with a higher degree of oxidation, by hydrogenation of the same and distillation.

N-Alkyl-substituted pyrrolidones are produced industrially by reacting alkylamines with gamma-butyrolactone. Thus, for example, the reaction of methylamine with gamma-butyrolactone gives N-methylpyrrolidone (NMP), which is used widely in industry as a solvent, as described by K. Weissermel, H.-J. Arpe in Industrielle Organische Chemie [Industrial Organic Chemistry], 5th edition, Wiley-VCH, page 115. Recovery of the NMP in pure form takes place by distillatively separating off any excess methylamine and also water of reaction. The purities which can be achieved are very high and are considerably more than 99.5%, usually above 99.8%. Other alkylamines, such as e.g. N-ethylpyrrolidone, are produced analogously.

When using N-alkylpyrrolidones, e.g. as solvents, these are changed in their composition, which impedes direct re-use although this would be very desirable for environmental and cost reasons.

One application of N-alkyl-substituted pyrrolidones and in particular NMP is the manufacture of lithium ion batteries as solvents for the coatings of the anode and cathode carriers. For this, specific requirements are placed on the solvent; in particular the quality used should not fluctuate significantly and comprise no new secondary components not tested beforehand as to their influence on the process. For example, the metal ion contents should be below 5 ppb, water contents below 300 ppm, the total purity (inclusive of 1,3- and 1,4-dimethylpyrrolidione) above 99.8% (so-called “electronic grade quality”). After the first use of N-alkyl-substituted pyrrolidones, as well as the increase in the water content, contamination can arise e.g. as a result of chemical reaction, which prevents a desirable direct re-use of the N-alkyl-substituted pyrrolidone and in particular use in the manufacture of lithium ion batteries.

WO 2011/030728 A1 describes a distillation of NMP. As comparative example 1 in this application shows, in which NMP comprises contaminants which have a higher degree of oxidation than NMP itself, a distillation as described in WO 2011/030728 A1 cannot be used to achieve the required degrees of purity of the originally used NMP again. Degrees of purity like the freshly used N-alkylpyrrolidone can therefore not be achieved by means of pure distillation and are thus unsuitable for the recycling of N-alkyl-substituted pyrrolidones which are used in the manufacture of lithium ion batteries.

For the purification of NMP, the company AMCEC suggests on the Internet site http://www.amcec.com/nmp%20recovery.html (“AMCEC NMP Recovery System”) that contaminants can be removed by filtration over activated carbon or zeolites. However, according to comparative example 2 carried out in this application, N-alkylpyrrolidones such as NMP which comprises contaminants with a higher degree of oxidation than NMP cannot be purified in this way. Consequently, this purification by filtration is also unsuitable for the use of the N-alkyl-substituted pyrrolidone as solvent in battery manufacture with electronic grade quality.

It is therefore the object of the present invention to provide a method which makes it possible to recover, in a simple manner, efficiently and cost-effectively but with high yields, N-alkyl-substituted pyrrolidones in high degrees of purity which correspond to the electronic grade.

This object is achieved by a method for purifying N-alkyl-substituted pyrrolidones which comprise one or more of the contaminants of the formula I to VII,

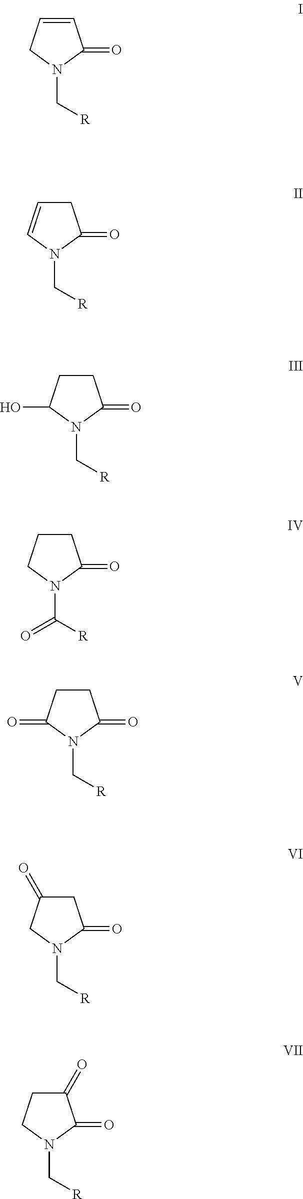

where R is selected from the group of hydrogen, linear or branched C₁-C₂₀-alkyl groups,
comprising the following steps
I) provision of a mixture comprising at least one N-alkyl-substituted pyrrolidone, at least one compound of the formula Ito VII in amounts of from 1 to 20 000 ppm
II) hydrogenation of the mixture from step I)
III) distillation of the mixture obtained from step II).

In the contaminants of the formula Ito VII, the radical R is hydrogen, linear or branched C₁-C₂₀-alkyl radicals. Preferably, R is hydrogen or a linear or branched C₁-C₁₀ alkyl radical. R is particularly preferably selected from the group of hydrogen, methyl, ethyl, n-propyl and n-butyl. R is very particularly preferably selected from the group of hydrogen and methyl. R is especially very particularly preferably hydrogen.

The amount of these contaminants of the formula I to VII in the N-alkyl-substituted pyrrolidone is generally, individually or as a mixture of some or all, in the range from 1 to 20 000 ppm, preferably between 2 and 15 000 ppm, particularly preferably between 5 and 10 000 ppm, based on the amount of mixture to be purified. In the N-alkyl-substituted pyrrolidone to be purified, in addition to the contaminants of the formula Ito VII, also their alkyl-substituted derivatives or oxo-bridged contaminants, individually or as a mixture of some or all, can be present. In the contaminants, the radical R1 is linear or branched C₁-C₂₀-alkyl radicals. Preferably, R1 is a linear or branched C₁-C₁₀ alkyl radical. R1 is particularly preferably selected from the group methyl or ethyl.

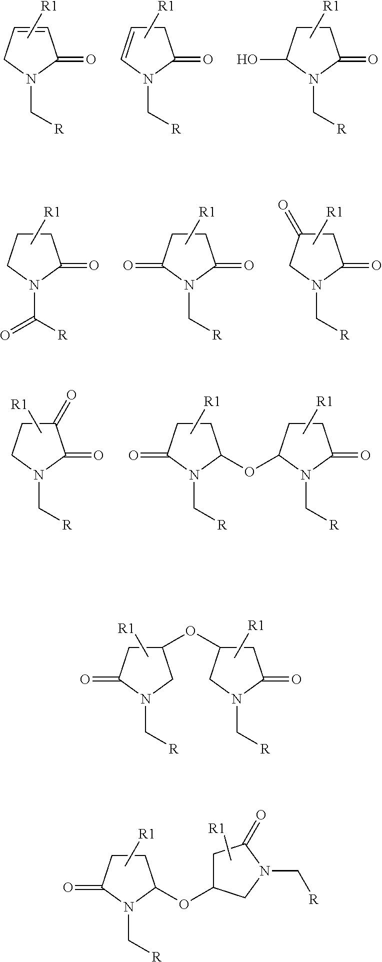

The water content of the N-alkyl-substituted pyrrolidone to be purified is generally between 0.05% and 90%, preferably between 0.1% and 50% and particularly preferably between 0.2%-10%.

The hydrogenation according to step II) of the method according to the invention can take place with the help of hydrogen and hydrogenation-active catalysts or through the use of complex hydrides.

In the case of hydrogenation or reduction by means of complex hydrides, those which are given in Advanced Organic Chemistry, J. March, 3rd edition, J. Wiley & Sons, 1985, pages 809-814 are preferably selected. Particular preference is given to sodium borohydride and lithium aluminum hydride.

The complex hydrides are added for example in amounts between 5 and 50 000 ppm, preferably between 50 and 25 000 ppm, particularly preferably between 100 and 10 000 ppm, based on the amount of mixture to be purified. The amount of complex hydrides is determined by the amount of contaminant; the complex hydride should be used at least stoichiometrically, but preferably superstoichiometrically relative to the amount of contaminant.

The complex hydrides can be introduced without a diluent or in solution or in the form of a suspension. Preferred solvents are water, provided it does not react with the hydride, the

N-alkyl-substituted pyrrolidone to be produced, ethers such as e.g. tetrahydrofuran and diethyl ether. Particular preference is given to the N-alkyl-substituted pyrrolidone to be produced. When using NaBH4, water is used particularly preferably as solvent.

The addition of the complex hydride which is added to the contaminated N-alkyl-substituted pyrollidone, in particular to the contaminated NMP, can take place at temperatures between 10 and 350° C. Preference is given to the method according to the invention when the temperature and residence time of the complex hydride is matched to one another. In general, the higher the temperature, the shorter the necessary residence time which is required to obtain the desired purifying effect. Thus, the residence times are for example from 0.1 to 10 hours at temperatures between 50 and 250° C., where the temperature does not have to be kept constant during the method according to the invention. The required residence time for the process is composed of the residence time in which the contaminated N-alkyl-substituted pyrrolidone is reacted together with the complex hydride and the residence time which is set during the distillation. If the residence time during the distillation is already sufficient, a separate residence-time step can be omitted.

The distillation can take place discontinuously or continuously. In this connection, all known distillation columns for the person skilled in the art can be used.

For example, when using a batchwise process, the procedure can be as follows: In a first step, the contaminated N-alkyl-substituted pyrrolidone, in particular NMP, is placed as initial charge together with the complex hydride in a stirred reactor, preferably in an inert-gas atmosphere, the substances are mixed e.g. by stirring or circulatory pumping and heated, optionally under pressure, to the desired temperature and the mixture is left to react. Then, in the second step, the contents are pumped into one or more columns and low-boiling components such as e.g. water and high-boiling components, which optionally comprise the complex hydride added in excess and any high-boiling components formed, are separated off (batchwise or continuously) from the N-alkyl-substituted pyrrolidone.

The first step described above can also proceed in a column before distillation is carried out.

The first step described above can likewise be carried out continuously. For this, in turn, a separate container upstream of the distillation or a distillation column can be used.

A preferred embodiment of the method involves adding the contaminated N-alkyl-substituted pyrrolidone and in particular the contaminated NMP and a complex hydride to a reaction product which is formed by reacting gamma-butyrolactone with methylamine to give NMP.

This mixture generally comprises N-alkyl-substituted pyrrolidone, water and the corresponding amine. This process variant is preferably carried out continuously. For this, in a first column, a mixture of amine, in particular methylamine for the purification of NMP, and water is separated off overhead (column pressure 300-5000 mbar, bottom temperatures 100-250° C. for the purification of contaminated NMP), the bottom product passes to a second column (column pressure 20-500 mbar, bottom temperatures 120 to 230° C., for the purification of contaminated NMP), in which remains of water and amine, in particular methylamine for the purification of NMP, are separated off overhead and the bottom product passes to a third column (column pressure 20-300 mbar, bottom temperatures 120 to 230° C., for the purification of contaminated

NMP), in which pure N-alkyl-substituted pyrrolidone, in particular NMP, is separated off overhead or via a side take-off while high-boiling components are discharged from the system via the bottom.

For this distillation sequence there are also variants such as e.g. that in the second column water and amine, in particular methylamine for the purification of NMP, are discharged overhead, N-alkyl-substituted pyrrolidone, in particular NMP, is discharged via the side take-off, and high-boiling components are discharged via the bottom. The N-alkyl-substituted pyrrolidone, in particular NMP, can then be further purified in a third column, as described, depending on the purity requirements. If the second column is configured as a dividing-wall column, then the N-alkyl-substituted pyrrolidone, in particular NMP, can be obtained already in very pure form as side take-off and a third column can be dispensed with.

The apparatuses used are manufactured from customary stainless steels, as are all of the pipelines and column internals such as packing, etc. In order to obtain N-alkyl-substituted pyrrolidones, in particular NMP, in “Electronic-grade” quality, it is advisable to also design the tanks used in stainless steel and to blanket them with protective gas such as e.g. nitrogen. Optionally, the N-alkyl-substituted pyrrolidone, in particular NMP, is additionally also passed over ion exchangers to remove metal ions and/or free amine traces.

A further process variant is the hydrogenation with hydrogen. The hydrogenation with hydrogen takes place in the presence of hydrogenation-active catalysts. These catalysts contain elements of the Periodic Table of the Elements selected from the group of cobalt, rhodium, ruthenium, iridium, nickel, palladium, platinum, copper, silver, gold or rhenium. Particular preference is given to the elements selected from the group of nickel, ruthenium, palladium, platinum and copper. The catalysts comprise the elements in the form of their metals and/or in the form of insoluble compounds, e.g. oxides, or are homogeneously soluble metal complexes.

The hydrogenation catalysts can be used as homogeneous soluble catalysts or as heterogeneous catalysts. If a homogeneously soluble catalyst is used, then this preferably comprises ruthenium. Particular preference is given to the use of heterogeneous catalysts.

The possible elements which can be used in the heterogeneous catalysts can be used individually and/or as a mixture. They can be used, for example, as unsupported, impregnated or precipitated catalysts, with or without dopants. Examples of supports or backbone materials of the catalysts are carbons such as activated carbon or graphite, and oxides of aluminum, silicon, titanium, cerium or zirconium. The fraction of hydrogenation element in the catalysts can be between 0.001 and 90% by weight. In the case of impregnated catalysts, the contents are preferably 0.001 to 20% by weight, particularly preferably 0.01 to 10% by weight, especially preferably 0.1 to 5% by weight. Very particularly preferred heterogeneous catalysts are Pd, Pt, Ru, Cu on activated carbon.

Preferably, the catalytic hydrogenation with regard to pressure and temperature should be carried out under the mildest conditions possible so that the N-alkyl-substituted pyrrolidone, in particular the NMP, is not itself hydrogenated, e.g. to the pyrrolidine, which would constitute a loss. Consequently, although pressures up to 320 bar are possible, they are preferably up to 100 bar and particularly preferably up to 50 bar. The lower limit is preferably at atmospheric pressure. The temperatures are generally 20 to 250° C., preferably 20 to 200° C., particularly preferably 20 to 150° C.

The heterogeneously catalyzed hydrogenation can be carried out in a fixed bed or in suspension. Preference is given to fixed bed in trickle or liquid-phase mode with or preferably without liquid circulation. The feed is passed together with hydrogen over the catalyst bed. The reaction product is firstly decompressed to the pressure level which is established in the subsequent distillation.

The conditions for the distillation step III) according to the invention are the same as for the hydrogenation by means of complex hydrides used.

### EXAMPLES

The examples below are intended to serve to illustrate the invention. The contents of contaminants given in the examples are determined by gas chromatography (GC instrument HP6890, FID detector, nitrogen carrier gas with 1.0 ml/min (const. flow); split ratio 1:50; column RTX-1, 30 m, 0.32 mm, 1.0 μm film; temperature program start at 80° C., then 5° C./min up to 140° C., then 5° C./min up to 200° C. and 10 min isotherm, then 10° C./min up to 340° C. and 8 min isotherm). All experiments were carried out under a nitrogen atmosphere. Water values were determined by means of Karl-Fischer titration. NMP and the corresponding contaminants were used as given in the examples.

### Comparative example 1

Distillation, as described in WO-A1 2011/030728.

A contaminated NMP, with 3% water and 500 ppm contaminants selected from the group of dehydro-NMP (contaminant of the formula I), oxo-NMP (contaminant of the formula VI) was subjected to fractional distillation in a 110 cm-long packed column (Sulzer pack) with reflux divider at bottom temperatures between 130 and 140° C. and pressures between at the start 1000 mbar (water separation) and 90 mbar top pressure at a reflux to take-off ratio of 50:1. Starting from an initial charge of 2 kg, a total of 16 distillate fractions were taken. The first two fractions, which were obtained at top temperatures of 55-118° C., contained predominantly water (>98%). At 100 mbar and a top temperature of 122-126° C., a fraction with 0.7% water and NMP at more than 99% was obtained. The subsequent fractions 4 to 13 (together ca. 1.5 kg) comprised water below 1000 ppm, NMP in purities above 99.8%, but still the contaminants already present in the initial charge. From fraction 14 onwards (together ca. 300 g), the NMP was free from the undesired contaminants. The experiment shows that by means of distillation alone, only an uneconomically small fraction of NMP (ca. 15%) can be recovered as NMP with the desired purity.

### Comparative Example 2

The starting material as in comparative example 1 was filtered over 500 ml of activated carbon and then distilled as in comparative example 1. Even after the filtration, the GC analysis showed that no depletion effect of the organic contaminants had occurred and also the distillation result was analogous to comparative example 1.

### Comparative Example 3

Comparative example 2 was repeated but using a zeolite instead of activated carbon. The result was the same as in example 2.

### EXAMPLES ACCORDING TO THE INVENTION

### Example 1

The feed mixture from comparative example 1 was admixed with 1000 ppm of NaBH₄ and heated at 105° C. for 0.5 h. According to gas chromatographic analysis, the undesired contaminants could no longer be detected. Subsequent distillation as in comparative example 1 produced ca. 95% NMP in the desired purity, with the majority of the remainder only being contaminated by excessively high water contents (>1000 ppm).

### Example 2

Example 1 was repeated except that the contaminant was N-methylsuccinimide (contaminant V, R=H) with 1000 ppm. The result was the same.

### Example 3

300 mL of the feed mixture from example 1 were admixed in an autoclave with 1% by weight of palladium (5% by weight) on activated carbon, 10 bar of hydrogen were injected, and the mixture was heated at 100° C. for 5 h. After cooling, decompressing and filtering off the catalyst, the mixture was worked up as in example 1. This resulted in ca. 95% of pure NMP without the contaminants of the formula I and VI.

### Example 4

Example 3 was repeated except that the catalyst used was nickel (10% by weight) on aluminum oxide and the hydrogenation was carried out at 130° C. Again, this resulted in ca. 95% of pure NMP without the contaminants of the formula I and VI.

### Example 5

Analogously to example 2, N-ethylpyrrolidone which was contaminated with 500 ppm of N-ethylsuccinimide (contaminant V, R=Me) was used. The amount of NaBH₄ added was 1000 ppm. More than 95% pure N-ethylpyrrolidone was obtained which no longer comprised N-ethylsuccinimide.

We claim:

1. A method for purifying N-alkyl-substituted pyrrolidones which comprise one or more of the contaminants of the formula Ito VII, 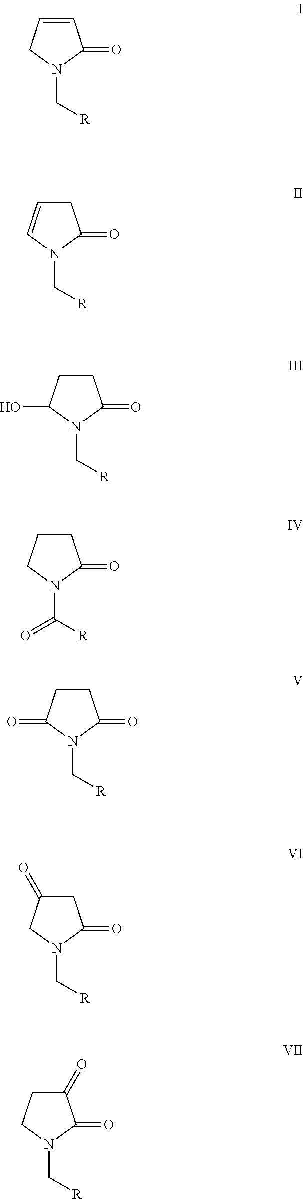
where R is selected from the group hydrogen, linear or branched C₁-C₂₀-alkyl groups,
comprising the following steps
I) provision of a mixture comprising at least one N-alkyl-substituted pyrrolidone, at least one compound of the formula Ito VII in amounts of from 1 to 20 000 ppm
II) hydrogenation of the mixture from step I)
III) distillation of the mixture obtained from step II).

2. The method according to claim 1, where the hydrogenation in step II) takes place either by means of hydrogen and a hydrogenation catalyst or by means of a complex hydride.

3. The method according to either of claims 1 and 2, where the complex hydrides are selected from the group of sodium borohydride and lithium aluminum hydride.

4. The method according to any one of claims 1 to 3, where the complex hydride is added in amounts of from 5 to 50 000 ppm, based on the amount of mixture to be purified from step I) of the method according to the invention.

5. The method according to any one of claims 1 to 4, where the contaminant of the formula I to VII are in contact during step II and/or step III with the complex hydride at optionally varying temperatures in the range from 50 to 250° C. over a period from 0.1 to 10 hours.

6. The method according to either of claims 1 and 2, where the metal of the hydrogenation catalyst is selected from the group of cobalt, rhodium, ruthenium, iridium, nickel, palladium, platinum, copper, silver, gold and rhenium.

7. The method according to claim 6, where the pressure during the hydrogenation in step II) is in the range from standard pressure up to 320 bar and the temperature is in the range from 20 to 250° C.

8. The method according to either of claims 6 and 7, where, for step II), processing is in the presence of a heterogeneous hydrogenation catalyst.

9. The method according to any one of claims 1 to 8, where the distillation in step Ill) is carried out continuously.

10. The method according to any one of claims 1 to 9, where the distillation in step Ill) takes place in at least two columns.

11. The method according to any one of claims 1 to 10, where the N-alkyl-substituted pyrrolidone is N-methylpyrrolidone.

12. The method according to claim 9, in which the distillation in step Ill) is carried out at temperatures in the range from 100 to 250° C. and pressures in the range from 300 to 5000 mbar.
